(19) United States
(12) Patent Application Publication
(10) Pub. No.: US 2025/0204793 A1
(43) Pub. Date: Jun. 26, 2025

(54) METHOD FOR VITAL MONITORING SYSTEM AND VITAL MONITORING SYSTEM

(71) Applicant: Harman International Industries, Incorporated, Stamford, CT (US)

(72) Inventors: Rengao Zhou, Shanghai (CN); Hanyu Shang, Shanghai (CN)

(21) Appl. No.: 18/848,074

(22) PCT Filed: Mar. 17, 2022
(86) PCT No.: PCT/CN2022/081422

§ 371 Date: Sep. 17, 2024

(51) Int. Cl. A61B 5/0205; A61B 5/021; A61B 5/024; A61B 5/145

(52) U.S. Cl. CPC A61B 5/0205; A61B 5/021; A61B 5/02405; A61B 5/14532; A61B 2503/22

(57) ABSTRACT

A method adapted for a vital monitoring system and the vital monitoring system are provided. The method obtains a current data block for data processing; searches the data block for vital data consisting of multiple pulse parts and stable parts, wherein the pulse parts and stable parts are interleaved; obtains a first interval and a second interval, wherein the first interval is an interval between two consecutive pulse parts at the beginning of the current data block, and wherein the second interval is an interval between two consecutive pulse parts at the end of the current data block; compares the first interval and the second interval; and alters a step size for stepping current data block forward, based on the comparison.

### TECHNICAL FIELD

The present disclosure relates to a method adapted for a vital monitoring system (VMS) and the VMS using the method, and specifically relates to the method for reducing the latency of the VMS by altering the step size of input data blocks.

### BACKGROUND

The VMS, Vital Monitoring System, is a concept mostly being used in medical fields. Recently, it has become popular to use VMS in the field of an Advanced Driver Assistance System (ADAS). People tend to use the VMS in the ADAS, in order to substantially reduce driver errors caused by distraction, drowsiness, or other abnormal vital conditions, such as hypoglycemia, heart abnormalities, hypertension, dehydration, and so on. Various sensors can be used in the VMS, including wearable IoT (Internet of Things) devices, RGB/IR cameras, biosensors and so on.

A wide variety of data, for example, including heart rate, heart rate variability, blood pressure, blood glucose level, blood alcohol concentration, may be collected or calculated by using various sensors. Usually, data is gathered over a long period and then combined into a data block. Data blocks are gathered, and one by one, will be analyzed. After analyzing and obtaining the vital situation, the smart vehicle may choose to take actions to improve the mental state of the driver accordingly. Actions may include changing cabin environment, playing music/sound, giving a scent, and so on. However, before analyzing and obtaining the vital situation, waiting until all necessary data is collected introduces latency. The latency might lead to a series of problems in case of severe driving situations.

Therefore, it is necessary to provide an improved technology to solve the latency issue.

### SUMMARY

According to one aspect of the disclosure, a method adapted for a vital monitoring system is provided. The method may comprise obtaining a current data block for data processing; searching the current data block for vital data consisting of multiple pulse parts and stable parts, wherein the pulse parts and stable parts are interleaved; obtaining a first interval and a second interval, wherein the first interval is an interval between two consecutive pulse parts at the beginning of the current data block, and wherein the second interval is an interval between two consecutive pulse parts at the end of the current data block; comparing the first interval and the second interval; and altering a step size for stepping current data block forward, based on the comparison.

According to another aspect of the present disclosure, a vital monitoring system is provided. The vital monitoring system may comprise a plurality of sensors and a processor. The plurality of sensors may be configured to capture data associated with a driver. The processor may be coupled to the plurality of sensors. The processor may be configured to obtain a current data block for data processing based on the data captured by the plurality of sensors, and search the current data block for vital data consisting of multiple pulse parts and stable parts, wherein the pulse parts and stable parts are interleaved. The processor may be configured to obtain a first interval and a second interval, wherein the first interval is an interval between two consecutive pulse parts at the beginning of the current data block, and wherein the second interval is an interval between two consecutive pulse parts at the end of the current data block. The processor may further be configured to compare the first interval and the second interval, and alter a step size for stepping current data block forward based on the comparison.

According to yet another aspect of the present disclosure, a non-transitory computer-readable storage medium comprising computer-executable instructions is provided which, when executed by a computer, causes the computer to perform the method disclosed herein.

### BRIEF DESCRIPTION OF THE DRAWINGS

FIG. 1 illustrates two cases of stepping the images data block.

FIG. 2 illustrates a flowchart of the method according to one or more embodiments of the present disclosure.

FIG. 3 illustrates an example of the vital data.

FIG. 4 illustrates a method flowchart to show how to get the two consecutive pulse parts at the beginning and end of the current data block according to one or more embodiments of the present disclosure.

FIG. 5 illustrates a flowchart of the method of altering the step size according to one or more embodiments of the present disclosure.

FIG. 6 illustrates a flowchart of a detailed method according to one or more embodiments of the present disclosure.

To facilitate understanding, identical reference numerals have been used, where possible, to designate identical elements that are common to the figures. It is contemplated that elements disclosed in one embodiment may be beneficially utilized on other embodiments without specific recitation. The drawings referred to here should not be understood as being drawn to scale unless specifically noted. Also, the drawings are often simplified and details or components omitted for clarity of presentation and explanation. The drawings and discussion serve to explain principles discussed below, where like designations denote like elements.

### DETAILED DESCRIPTION OF THE PREFERRED EMBODIMENTS

Examples will be provided below for illustration. The descriptions of the various examples will be presented for purposes of illustration, but are not intended to be exhaustive or limited to the embodiments disclosed. Many modifications and variations will be apparent to those of ordinary skill in the art without departing from the scope and spirit of the described embodiments.

As described above, the algorithms of VMS use a long period for data collection for analyzing the vital situation of the driver. For example, the algorithms will need a total of 512 RGB-camera-collected images for calculation. Assuming the FPS (frame per second) of images being 30, then collecting 512 images would take around 17 seconds. After analyzing this 512 images data block, it will again wait for the next whole 512 images to be collected before calculating. As a result, an average latency of 8.5 seconds would be introduced. And 8.5 seconds latency might lead to a series of problems in case of severe driving situations.

An alternative method may be to do the calculation upon the arrival of every single image. Instead of calculating a data block of 512 images and then waiting for the next data block, this alternative method cuts down the latency to a very small number. However, this method requires a lot of computing power, since a lot of complicated calculations, including neural networks, exist in the algorithms. Furthermore, it might not be necessary to perform calculations every time a new image arrives, because the difference between images might be considered too small. With this alternative method, only the first and last image of the 512 images data block would be changed, and most of the remaining 512 images are the same. FIG. 1 illustrates the two method described above. In FIG. 1, ‘CASE 1’ 101 is gathering the next new 512 images in a data block (i.e., frame sequence 2) after using the previous one (i.e., frame sequence 1). ‘CASE 2’ 102 is adding one new image at the end and deleting one old image at the beginning. This means the step size of ‘CASE 1’ 101 is 512 frames, while the step size of ‘CASE 2’ 102 is 1 frame.

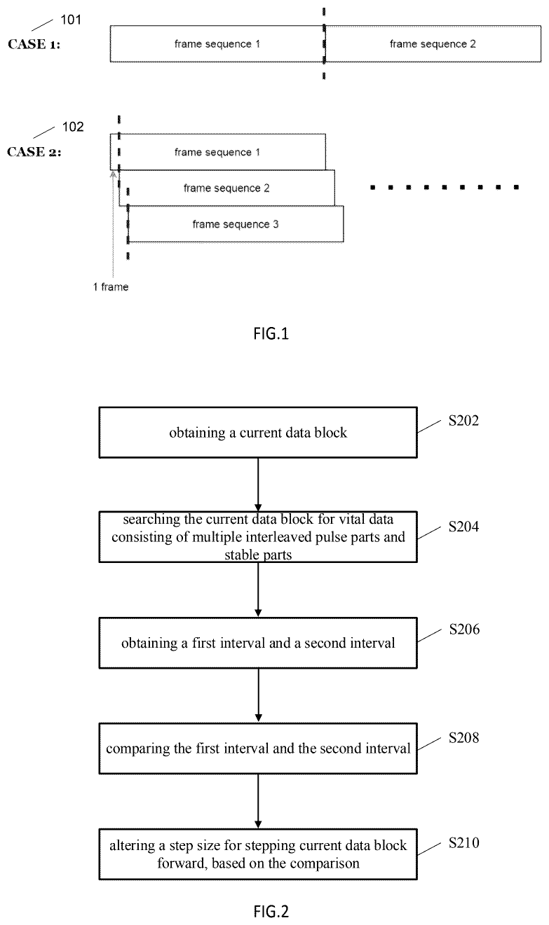

In the present disclosure, an improved method and system may be provided to address latency issues while requiring only a reasonable amount of computing power and ensuring that data blocks contain sufficient information for various analytical processing. This is achieved by flexibly altering the step size of input data blocks during the data processing in the VMS, instead of using a fixed and predetermined step size of data blocks. The innovation proposed in this disclosure may reduce the latency, save computing power, thereby obtaining the dynamic balance among the computing accuracy, computing power, and the latency for data processing in the VMS. The approach will be explained in detail referring to FIGS. 2-6 as follows.

FIG. 2 illustrates a flowchart of the method according to one or more embodiments of the present disclosure. At S202, the current data block may be obtained. For example, a plurality of sensors in the VMS may collect or calculate multiple data regarding different vital signs of the driver, such as data of heart beat, heart rate, heart rate variability, blood pressure, blood pulse, blood glucose level, blood alcohol concentration, and so on. The data may be combined into data block with a certain length to be processed by the algorithm used in the VMS.

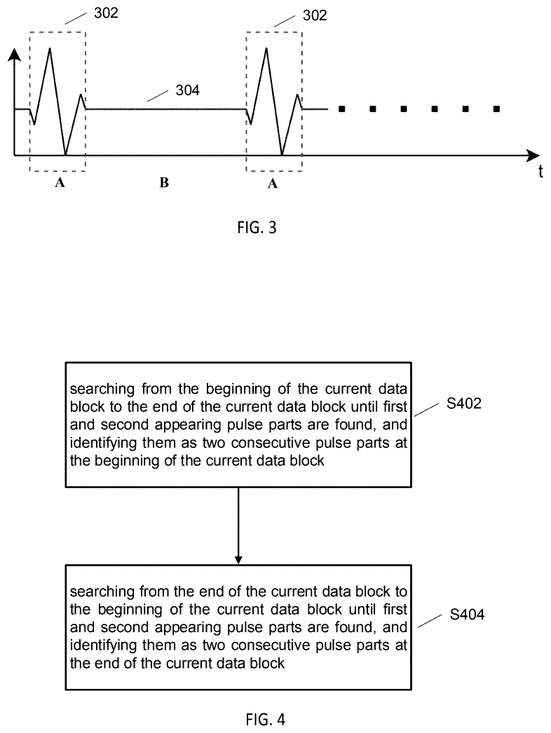

At S204, a searching procedure of the current data block is performed. The current data block is searched for vital data that is representative of a driver's vital signs and has characteristic of periodicity. For example, the vital data may consist of multiple pulse parts and stable parts that are interleaved. FIG. 3 illustrates an example of the vital data having the abovesaid characteristic, such as heart beat data. As shown in FIG. 3, the data regarding heart beat exhibit pulse and stable parts A 302 and B 304, the parts A 302 and B 304 are interleaved, such as an arrangement of A, B, A, B, A . . . Parts A 302 marked by dashed rectangles show ‘pulse waves’ resulting from heartbeats. And it can be noticed that, apart from the parts of pulse waves resulting from the heartbeats, other parts B 304 are roughly the same. For clear definition, parts A may be called the pulse parts, or may be called heart beats. Parts B may be called stable parts. FIG. 3 is merely an example of an ideal plot shown to illustrate the principle. Those skilled in the art can understand that the data generated based on the actual heartbeat may not be as perfect as shown in FIG. 3. Rather, the waves in each part A are usually not completely consistent, and part B may also include small fluctuations. However, this does not destroy the characteristic of the data as a whole. In practical operation, for example, a threshold or a threshold interval of the peak amplitude can be predefined according to experience. According to whether the relationship between the peak value of the actual data and the pre-defined threshold or threshold interval satisfies certain criteria, data with desired characteristics that are being looked for can be determined, that is, the vital data consisting of pulse parts and stable parts that are interleaved can detected.

At S206, based on the searched vital data, a first interval and a second interval may be obtained respectively. The first interval may be obtained by calculating an interval between two consecutive pulse parts at the beginning of the current data block. The second interval may be obtained by calculating an interval between two consecutive pulse parts at the end of the current data block.

Taking the heart beat as an example, the speed of heart beat, or to say, the frequency of heart beat, depends on the time interval between two consecutive heart beats. The first interval and a second interval are looked at respectively at the beginning and the end of the data block because the heart beat frequency at the beginning of data block might be different from the heart beat frequency at the end of the data block.

There are various approaches to get the value of interval between two consecutive heart beats. For example, the frequency of a heart beat may be directly retrieved from the detected value from a wearable IoT (Internet of Things) device. Also, the frequency of heart beat could be derived from the RGB camera images, using algorithms. Other methods may also be implemented for obtaining the frequency of heart beat.

The interval between two consecutive heart beats may be obtained based on peak data representative of heart beats. For example, the interval between two consecutive peak data may be calculated, thereby the interval between two consecutive heart beats may be obtained.

At S208, the first interval representative of the interval between two consecutive pulse parts at the beginning of the current data block, may be compared with the second interval representative of the interval between two consecutive pulse parts at the beginning of the current data block. The first interval and the second interval may be in units of time or frames. The comparison result may be obtained.

At S210, based on the comparison result at S208, a step size for stepping data block forward may be altered.

According to the method in FIG. 2, the step size for stepping data blocks forward is not a fixed and predetermined value, but a flexible value that alters with the actual physical condition of a person. This method can closely combine data processing with actual physical signs, and achieve a dynamic balance between processing latency and computational power. In addition, the method uses the interval of two consecutive pulse parts as a basis for determining the step size, which ensures that sufficient information is included in the data block used for data processing, because most of the useful information for data (such as heart beats) is included in the pulse parts. Therefore, it is feasible to step the data block forward until reaching the next occurrence of heart beat. Although some data are skipped, those data are expected to contain very little useful information.

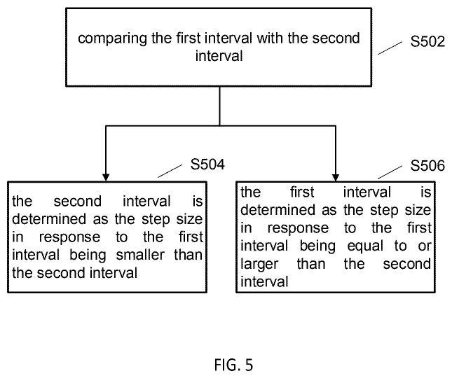

FIG. 4 illustrates a flowchart of a method for obtaining two consecutive pulse parts at the beginning and end of the current data block according to one or more embodiments of the present disclosure. At S402, the method searches from the beginning of the current data block to the end of the current data block until it finds the first and second occurrences of pulse parts, and identifies them as two consecutive pulse parts at the beginning of the current data block. At S404, the method searches from the end of the current data block to the beginning of the current data block until it finds the first and second occurrences of pulse parts, and identifies them as two consecutive pulse parts at the end of the current data block. S402 and S404 may be performed sequentially, in reverse order, or in parallel.

FIG. 5 illustrates a flowchart of the method of altering the step size according to one or more embodiments of the present disclosure. At S502, the first interval representative of the interval between two consecutive pulse parts at the beginning of the current data block may be compared with the second interval representative of the interval between two consecutive pulse parts at the end of the current data block. At S504, the second interval is selected as the step size for stepping data block forward in response to the first interval being smaller than the second interval. At S506, the first interval is selected as the step size for stepping data block forward in response to the first interval being equal to or larger than the second interval. The step size may be in units of time or frames.

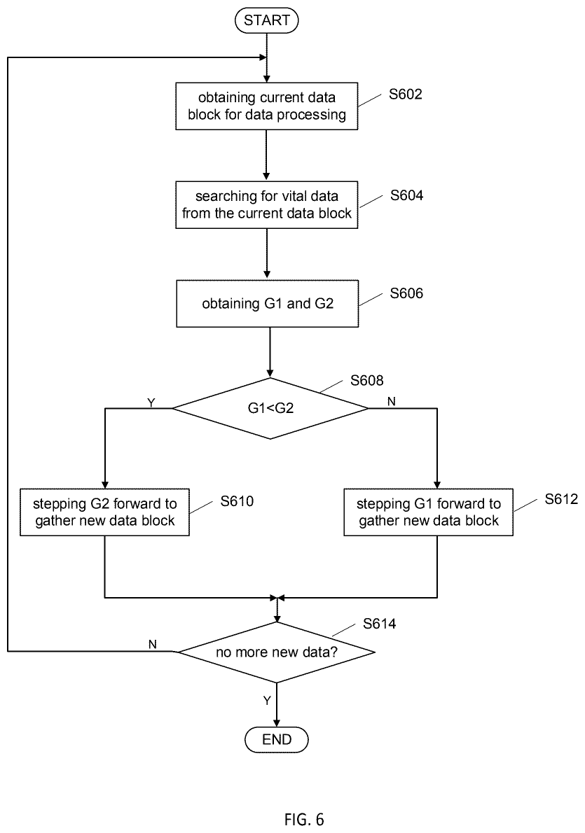

FIG. 6 illustrates a detailed example of the method according to one or more embodiments of the present disclosure. As shown in FIG. 6, at S602, current data block may be obtained. For example, a plurality of sensors in the VMS may collect or calculate data regarding different vital signs of the driver. The data may be combined into a data block with a predetermined length to be processed by the algorithm used in the VMS.

At S604, the searching procedure may be performed to look for vital data from the current data block. The vital data may be representative of a driver's vital signs and have a characteristic of periodicity, and the vital data may consist of multiple pulse parts and stable parts that are interleaved, as discussed in reference to FIG. 2-3. Details are similar to the descriptions corresponding to FIGS. 2-3, and omitted herein.

At S606, G1 and G2 may be obtained based on the searched vital data from the current data block. G1 is an interval between two consecutive pulse parts (e.g., heart beats) in vital data at the beginning of the current data block. G2 is an interval between two consecutive pulse parts (e.g., heart beats) in vital data at the end of the current data block. According to one or more embodiments, the two consecutive pulse parts at the beginning of the current data block are the first and second pulse parts obtained as searching from the beginning of the current data block to the end of the current data block. And, the two consecutive pulse parts at the end of the current data block are the first and second pulse parts obtained as searching from the end of the current data block to the beginning of the current data block.

At S608, a comparison may be performed to determine whether G1 is smaller than G2. If at S608 G1 is smaller than G2, then the method goes to S610. At S610, G2 is selected as the step size, i.e., stepping G2 forward to gather new data block, to ensure that a new ‘pulse’ would be included in the next selected data block. Then, the method goes to S614 to determine if there is no more new data. If there is no more new data, the method goes to the END. If there is more new data, the method returns to START.

If at S608 G1 is not smaller than G2, then the method goes to S612. At S612, G1 is selected as the step size, i.e., stepping G1 forward to gather a new data block. In this case, a new ‘pulse’ would most likely be assured, thus, it can be ensured that the old ‘pulse’ at the beginning of data block is dismissed. Then, the method goes to S614 to determine if there is no more new data. If there is no more new data, the method goes to the END. If there is more new data, the method returns to START.

The method described in this disclosure may be adapted for the vital monitoring system VMS. The VMS may comprise sensors and a processor coupled to the sensors. For example, the sensors may collect data associated with the driver. The processor may perform all the methods described above. For example, the processor may obtain current data block for data processing based on the data captured by the plurality of sensors, and search for vital data consisting of multiple pulse parts and stable parts from the current data block, wherein the pulse parts and stable parts are interleaved. The processor may obtain a first interval and a second interval, wherein the first interval is an interval between two consecutive pulse parts at the beginning of the current data block, and wherein the second interval is an interval between two consecutive pulse parts at the end of the current data block. Then, the processor may compare the first interval and the second interval, and alter a step size for stepping current data block forward based on the comparison.

The processor may be any technically feasible hardware unit configured to process data and execute software applications, including without limitation, a central processing unit (CPU), a microcontroller unit (MCU), an application specific integrated circuit (ASIC), a digital signal processor (DSP) chip and so forth.

1. In some embodiments, a method for a vital monitoring system (VMS), comprising: obtaining a current data block for data processing; searching the current data block for vital data consisting of multiple pulse parts and stable parts, wherein the pulse parts and stable parts are interleaved; obtaining a first interval and a second interval, wherein the first interval is an interval between two consecutive pulse parts at the beginning of the current data block, and wherein the second interval is an interval between two consecutive pulse parts at the end of the current data block; comparing the first interval and the second interval; and altering a step size for stepping current data block forward, based on the comparison.

2. The method according to clause 1, wherein the altering the step size for stepping data block forward based on the comparison comprises: selecting the second interval as the step size in response to the first interval being smaller than the second interval; or selecting the first interval as the step size in response to the first interval being equal to or larger than the second interval.

3. The method according to any one of clauses 1-2, wherein the two consecutive pulse parts at the beginning of the current data block are first and second pulse parts obtained as searching from the beginning of the current data block to the end of the current data block; and wherein the two consecutive pulse parts at the end of the current data block are first and second pulse parts obtained as searching from the end of the current data block to the beginning of the current data block.

4. The method according to any one of clauses 1-3, further comprising gathering new data block as next data block for data processing, based on the selected step size.

5. The method according to any one of clauses 1-4, wherein the vital data includes at least one of heart beat data and blood pulse data.

6. The method according to any one of clauses 1-5, wherein the vital data is searched out based on peaks data that occur periodically.

7. The method according to any one of clauses 1-6, wherein the VMS is used in an Advanced Driver Assistance System (ADAS).

8. In some embodiments, vital monitoring system (VMS), comprising: a plurality of sensors configured to capture data associated with a driver; and a processor coupled to the sensors and configured to: obtain a current data block for data processing based on the data captured by the plurality of sensors; search the current data block for vital data consisting of multiple pulse parts and stable parts, wherein the pulse parts and stable parts are interleaved; obtain a first interval and a second interval, wherein the first interval is an interval between two consecutive pulse parts at the beginning of the current data block, and wherein the second interval is an interval between two consecutive pulse parts at the end of the current data block; compare the first interval and the second interval; and alter a step size for stepping current data block forward based on the comparison.

9. The vital monitoring system according to clause 8, wherein the processor is configured to select the second interval as the step size in response to the first interval being smaller than the second interval; or select the first interval as the step size in response to the first interval being equal to or larger than the second interval.

10. The vital monitoring system according to any one of clauses 8-9, wherein the two consecutive pulse parts at the beginning of the current data block are the first and second pulse parts obtained as searching from the beginning of the current data block to the end of the current data block; and wherein the two consecutive pulse parts at the end of the current data block are the first and second pulse parts obtained as searching from the end of the current data block to the beginning of the current data block.

11. The vital monitoring system according to any one of clauses 8-10, wherein the processor is configured to gather new data block as next data block for data processing, based on the selected step size.

12. The vital monitoring system according to any one of clauses 8-11, wherein the vital data includes at least one of heart beat data and blood pulse data.

13. The vital monitoring system according to any one of clauses 8-12, wherein the vital data is searched out based on peaks data that occur periodically.

14. The vital monitoring system according to any one of claims 8-13, wherein the VMS is used in an Advanced Driver Assistance System (ADAS).

15. A computer-readable storage medium comprising computer-executable instructions which, when executed by a computer, causes the computer to perform the method according to any one of claims 1-7.

The descriptions of the various embodiments have been presented for purposes of illustration, but are not intended to be exhaustive or limited to the embodiments disclosed. The terminology used herein was chosen to best explain the principles of the embodiments, the practical application or technical improvement over technologies found in the marketplace, or to enable others of ordinary skill in the art to understand the embodiments disclosed herein.

In the preceding, reference is made to embodiments presented in this disclosure. However, the scope of the present disclosure is not limited to specific described embodiments. Instead, any combination of the preceding features and elements, whether related to different embodiments or not, is contemplated to implement and practice contemplated embodiments. Furthermore, although embodiments disclosed herein may achieve advantages over other possible solutions or over the prior art, whether or not a particular advantage is achieved by a given embodiment is not limiting the scope of the present disclosure. Thus, the preceding aspects, features, embodiments and advantages are merely illustrative and are not considered elements or limitations of the appended claims except where explicitly recited in a claim(s).

Aspects of the present disclosure may take the form of an entirely hardware embodiment, an entirely software embodiment (including firmware, resident software, micro-code, etc.) or an embodiment combining software and hardware aspects that may all generally be referred to herein as a “circuit,” “module”, “unit” or “system.”

The present disclosure may be a system, a method, and/or a computer program product. The computer program product may include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective calculating/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

The flowchart and block diagrams in the drawings illustrate the architecture, functionality, and operation of possible implementations of systems, methods, and computer program products according to various embodiments of the present disclosure. In this regard, cach block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

While the foregoing is directed to embodiments of the present disclosure, other and further embodiments of the disclosure may be devised without departing from the basic scope thereof, and the scope thereof is determined by the claims that follow.

1. A method for a vital monitoring system (VMS), comprising:
obtaining a current data block for data processing;
searching the current data block for vital data comprising multiple pulse parts and stable parts, wherein the pulse parts and stable parts are interleaved, wherein searching comprises identifying first consecutive pulse parts at a beginning of the current data block and second consecutive pulse parts at an end of the current data block;
determining a first interval and a second interval, wherein the first interval is an interval between the first consecutive pulse parts at the beginning of the current data block, and wherein the second interval is an interval between the second consecutive pulse parts at the end of the current data block;
comparing the first interval and the second interval;
altering a step size for stepping current data block forward, based on the comparison; and
obtaining a next data block for data processing according to the altered step size, wherein the step size is a difference between a first frame of the current data block and a first frame of a next data block.

2. The method according to claim 1, wherein the VMS comprises a plurality of sensors configured to obtain vital data of a driver of a vehicle.

3. The method according to claim 1, wherein the step size is one of a unit of time or a number of frames.

4. The method according to claim 1, wherein, based on the comparison, the first interval is smaller than the second interval, selecting the second interval as the altered step size.

5. The method according to claim 1, wherein, based on the comparison, the second interval is smaller than the first interval, selecting the first interval as the altered step size.

6. The method according to claim 1,
wherein searching for the first consecutive pulse parts at the beginning of the current data block compromises searching from the beginning of the current data block to the end of the current data block to identify first two consecutive pulse parts.

7. The method according to claim 1, wherein searching for the second consecutive pulse parts at the end of the current data block comprises searching from the end of the current data block to the beginning of the current data block to identify first two consecutive pulse parts.

8. The method according to claim 1, wherein the vital data includes at least one of heart beat data and blood pulse data.

9. (canceled)

10. The method according to claim 1, wherein the vital data is searched out based on peaks data that occur periodically.

11. The method according to claim 1, wherein the VMS is used in an Advanced Driver Assistance System (ADAS).

12. A vital monitoring system (VMS), comprising:
a plurality of sensors configured to capture data associated with a driver of a vehicle; and
a processor coupled to the plurality of sensors and configured to:
obtain a current data block for data processing based on the data captured by the plurality of sensors;
search the current data block for vital data comprising multiple pulse parts and stable parts, wherein the pulse parts and stable parts are interleaved, wherein searching comprises identifying first consecutive pulse parts at a beginning of the current data block and second consecutive pulse parts at an end of the current data block;
determine a first interval and a second interval, wherein the first interval is an interval between the first consecutive pulse parts at the beginning of the current data block, and wherein the second interval is an interval between the first consecutive pulse parts at the end of the current data block;
compare the first interval and the second interval;
alter a step size for stepping current data block forward based on the comparison; and obtain a next data block for data processing, wherein the altered step size is a difference in frames between the current data block and the next data block.

13. The vital monitoring system according to claim 12, wherein the processor is configured to:
select the second interval as the step size in response to the first interval being smaller than the second interval; and
select the first interval as the step size in response to the first interval being equal to or larger than the second interval.

14. The vital monitoring system according to claim 12,
wherein: the first consecutive pulse parts at the beginning of the current data block are first and second pulse parts obtained by searching from the beginning of the current data block to the end of the current data block; and
wherein the two consecutive pulse parts at the end of the current data block are the first and second pulse parts obtained as searching from the end of the current data block to the beginning of the current data block.

15. The vital monitoring system according to claim 12, wherein the current data block is a combination of data from the plurality of sensors over a certain time.

16. The vital monitoring system according to claim 12, wherein the vital data includes at least one of heart beat data and blood pulse data.

17. The vital monitoring system according to claim 12, wherein the vital data is searched out based on peaks data that occur periodically.

18. The vital monitoring system according to claim 12, wherein the VMS is used in an Advanced Driver Assistance System (ADAS).

19. A computer-readable storage medium comprising computer-executable instructions which, when executed by a computer, causes the computer to obtain data with a plurality of sensors of a vital monitoring system (VMS) incorporated into a vehicle over a defined time;
combine the data from the plurality of sensors over the defined time into a current data block, wherein the current data block comprises multiple interleaved pulse parts and stable parts;
determine first consecutive pulse parts at a beginning of the current data block by searching the current data block from beginning to end;
determine second consecutive pulse parts at an end of the current data block by searching the current data block from end to beginning;
determine a first interval between the first consecutive pulse parts and a second interval between the second consecutive pulse parts;
compare the first interval to the second interval;
in response to the first interval being smaller than the second interval, alter a step size between the current data block and a next data block based on the second interval;
in response to the second interval being smaller than the first interval, alter the step size between the current data block and the next data block based on the first interval; and
obtain the next data block according to the altered step size.

20. The computer-readable storage medium according to claim 19, wherein the altered step size is a difference in frames between a first frame of the current data block and a first frame of the next data block.

21. The computer-readable storage medium according to claim 19, wherein the data obtained with the plurality of sensors comprises one or more of heart beat data, heart rate data, heart rate variability data, blood pressure data, and blood glucose level data.
